(19) United States
(12) Patent Application Publication
(10) Pub. No.: US 2024/0138547 A1
(43) Pub. Date: May 2, 2024

(54) BOXING ASSISTANCE SLEEVE

(71) Applicant: Lisa Valencia, Los Angeles, CA (US)

(72) Inventor: Lisa Valencia, Los Angeles, CA (US)

(21) Appl. No.: 18/496,851

(22) Filed: Oct. 28, 2023

Related U.S. Application Data
(60) Provisional application No. 63/381,549, filed on Oct. 28, 2022.

(51) Int. Cl. A45F 5/00; A61F 17/00

(52) U.S. Cl. CPC A45F 5/00; A61F 17/00; A45F 2005/008

(57) ABSTRACT

A device, system and method of providing relevant tools and devices to athletic fighting coaches, players and teams for usage during training and competitions. The device, system and method are most intended to be utilized during intermissions or between rounds of a fighting competition. The device, method and systems provide quick and organized access to the tools and devices that athletic fighting coaches, players and teams commonly use.

### CROSS-REFERENCE

This application claims priority from the U.S. Provisional Patent Application with Ser. No. 63/381,549, entitled, “BOXING ASSISTANCE SLEEVE” filed on Oct. 28, 2022, which is incorporated herein by reference in its entirety.

### TECHNICAL FIELD

The present disclosure relates to athletic equipment. More particularly, the disclosure relates to an improved athletic assistance device in the field of boxing and related sports.

### BACKGROUND

A boxing match is often separated into rounds wherein between the rounds are intervals of rest periods. During these rest periods the boxer's coach or team will assess the fighter's physical condition, provide coaching advice and allow medical assistance such as epinephrine and or vaseline to attempt to stop or slow any bleeding that occurs during the fight.

A high performing boxing coach or team will have an array of tools available and the knowledge to optimize their usage while understanding these tools' inherent limitations. Furthermore, a high performing boxing coach or team will utilize the limited time interval available for the rest period. In many boxing competitions, these rest periods are limited to one minute. As such, the boxing industry is inherently looking for innovative devices, systems, and methods that allow the most optimal usage of these rest periods. Furthermore such devices, systems, and methods would be immensely beneficial for other similar sports industries such as Mixed Martial Arts (MMA). Within this application such devices, systems and methods are described and disclosed.

### SUMMARY

Aspects and advantages of the invention will be set forth in part in the following description, or may be obvious from the description, or may be learned through practice of the invention.

In general, one innovative aspect of the subject matter described in this specification can be embodied as a fabric wherein the fabric comprises of an external side, an internal side, a hole, with at least one fastening attachment mechanism, and a plurality of holders.

In some embodiments, the fabric comprises of an external side, an internal side, a hole, with at least one fastening attachment mechanism, and a plurality of holders wherein the plurality of holders are disposed onto the external side of the fabric and the fastening mechanism.

In some embodiments, the fabric comprises of an external side, an internal side, a hole, with at least one fastening attachment mechanism, and a plurality of holders wherein the plurality of holders comprises of at least one enswell holder.

In some embodiments, the fabric comprises of an external side, an internal side, a hole, with at least one fastening attachment mechanism, and a plurality of holders wherein the plurality of holders comprises of an at least one cylindrical holder.

In some embodiments, the fabric comprises of an external side, an internal side, a hole, with at least one fastening attachment mechanism, and a plurality of holders wherein the plurality of holders comprises of at least one enswell holder and an at least one cylindrical holder.

In some embodiments, the fabric comprises of an external side, an internal side, a hole, with at least one fastening attachment mechanism, and a plurality of holders wherein the plurality of holders comprises of at least one enswell holder, at least one gauze holder, at least one cotton swab holder, and an at least one cylindrical holder.

In some embodiments, the fabric comprises of an external side, an internal side, a hole, with at least one fastening attachment mechanism, and a plurality of holders wherein the size and orientation of the fabric enables the internal side of the fabric to wrap around the arm of a user and wherein the size and orientation of the fabric enables the user to place their thumb.

In some embodiments, the fabric comprises of an external side, an internal side, a hole, with at least one fastening attachment mechanism, and a plurality of holders wherein the fastening mechanism comprises of a hook-and-loop fastener.

In some embodiments, the fabric comprises of an external side, an internal side, a hole, with at least one fastening attachment mechanism, and a plurality of holders wherein the fastening mechanism comprises of a hook-and-loop fastener, wherein the hook-and-loop fastener is positioned on the external side of the fabric.

In some embodiments, the fabric comprises of an external side, an internal side, a hole, with at least one fastening attachment mechanism, and a plurality of holders wherein the plurality of holders comprises of at least one enswell holder, wherein the enswell holder further comprises of a cooling material.

In some embodiments, the fabric comprises of an external side, an internal side, a hole, with at least one fastening attachment mechanism, and a plurality of holders wherein the plurality of holders comprises of at least one enswell holder, wherein the enswell holder further comprises of a cooling material wherein the cooling material is lined within the external surface of the fabric.

In some embodiments, the fabric comprises of an external side, an internal side, a hole, with at least one fastening attachment mechanism, and a plurality of holders wherein the fabric is made of neoprene.

In some embodiments, the fabric comprises of an external side, an internal side, a hole, with at least one fastening attachment mechanism, and a plurality of holders wherein the fabric is partially made of neoprene.

In some embodiments, the fabric comprises of an external side, an internal side, a hole, with at least one fastening attachment mechanism, and a plurality of holders wherein the plurality of holders comprises of at least one enswell holder, wherein the enswell holder further comprises a sleeve and a protuberance.

In some embodiments, the fabric comprises of an external side, an internal side, a hole, with at least one fastening attachment mechanism, and a plurality of holders wherein the plurality of holders comprises of at least one enswell holder, wherein the enswell holder further comprises a plastic clip and a protuberance.

In some embodiments, the fabric comprises of an external side, an internal side, a hole, with at least one fastening attachment mechanism, and a plurality of holders wherein the plurality of holders comprises of an at least one cylindrical holder, wherein the at least one cylindrical holder is configured to hold a tin.

In some embodiments, the fabric comprises of an external side, an internal side, a hole, with at least one fastening attachment mechanism, and a plurality of holders wherein the plurality of holders comprises of an at least one cylindrical holder, wherein the at least one cylindrical holder is configured to hold a tin of grease.

In some embodiments, the fabric comprises of an external side, an internal side, a hole, with at least one fastening attachment mechanism, and a plurality of holders wherein the plurality of holders comprises of at least one enswell holder, at least one gauze holder, at least one cotton swab holder, and an at least one cylindrical holder, wherein the at least one cotton swab holder is positioned and oriented so that a cotton swab within the at least one cotton swab holder is held in a position parallel with the longest length of the fabric.

In some embodiments, the fabric comprises of an external side, an internal side, a hole, with at least one fastening attachment mechanism, and a plurality of holders.

In some embodiments, the fabric comprises of an external side, an internal side, a hole, with at least one fastening attachment mechanism, and a plurality of holders wherein the hole is positioned to fit around a user's thumb when the internal side of the fabric is wrapped around the arm of the user.

### BRIEF DESCRIPTION OF THE FIGURES

A full and enabling disclosure of the present invention, including the best mode thereof, directed to one of ordinary skill in the art, is set forth in the specification, which makes reference to the appended drawings in, which:

FIG. 1 is a top, right, perspective view of an embodiment of the invention, wherein the fabric (10) wherein the fastening attachment mechanism (15) is in the form of hook-and-loop fasteners, wherein the cylindrical holder (30) is holding a tin can embodied as a grease tin.

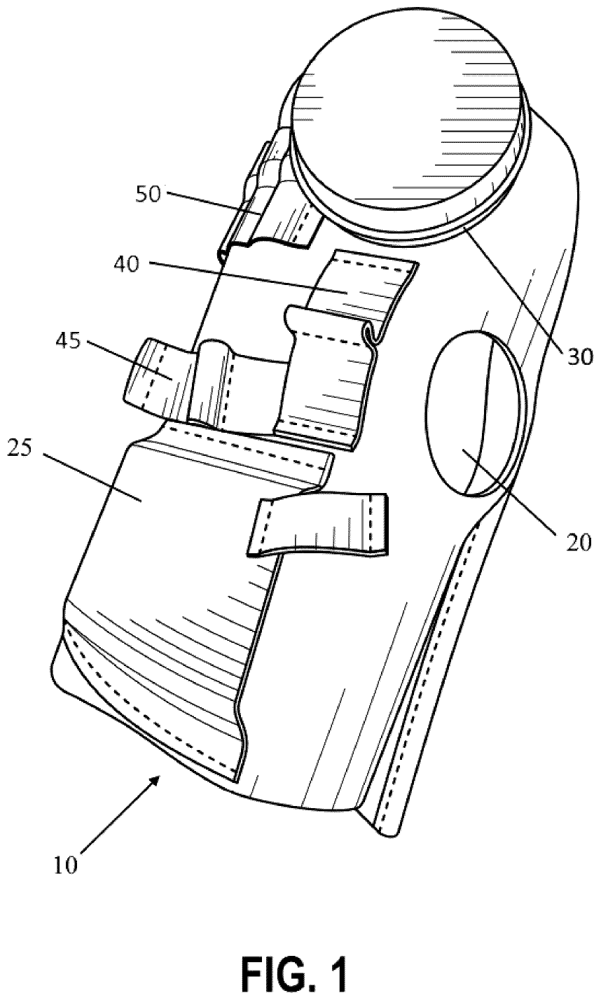

FIG. 2 is a top, right, perspective view of an embodiment of the invention, wherein the fabric (10) is shown positioned with a user's hand in dotted line form to demonstrate positioning when wearing the fabric (10), wherein the fastening attachment mechanism (15) is in the form of hook-and-loop fasteners, wherein the cylindrical holder (30) is holding a tin can embodied as a grease tin.

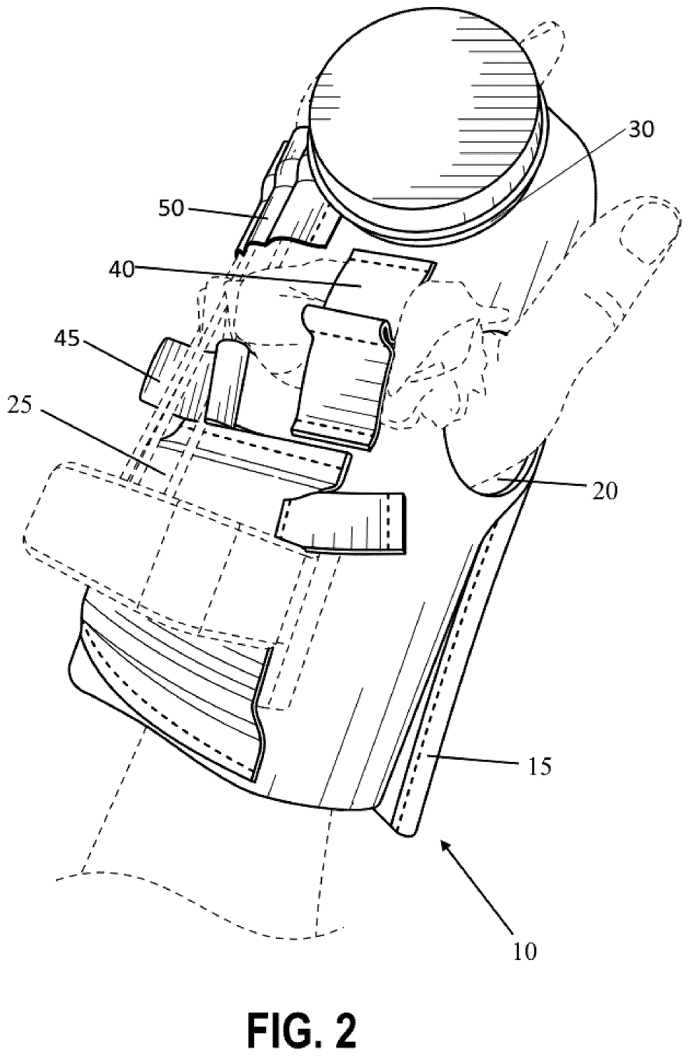

FIG. 3 is a top view of an embodiment of the invention wherein a gauze material holder (40), a gauze plug holder (45) and a cotton tip applicator holder (50) are clearly shown positioned substantially on the top side of the invention, and the wherein the lateral side of the cylindrical holder (30) visible.

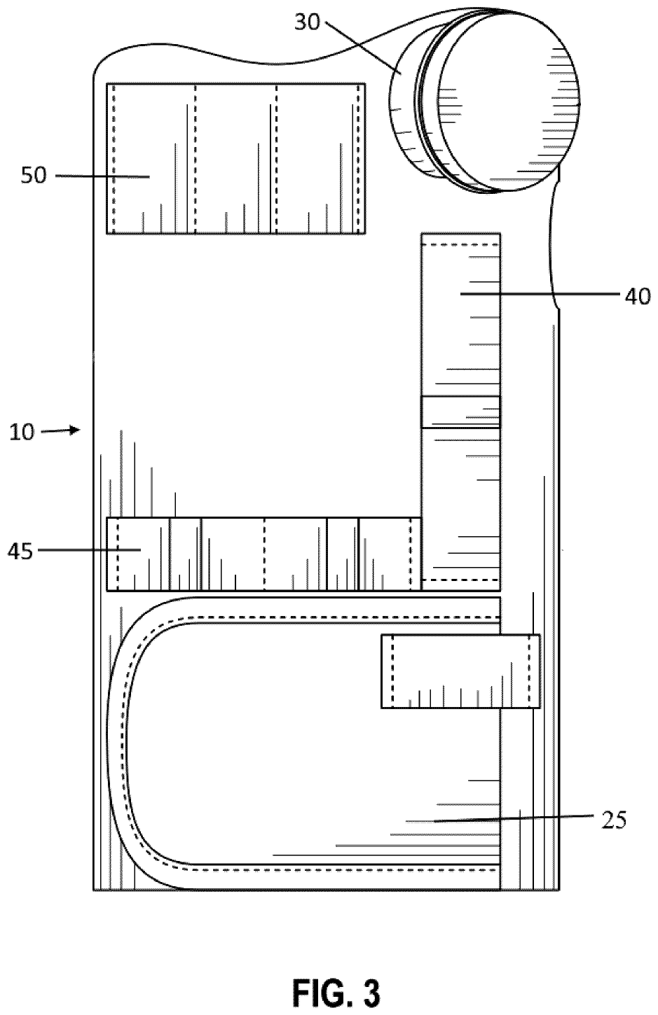

FIG. 4 is a bottom view of an embodiment of the invention wherein the fastening attachment mechanism (15) is in the form of hook-and-loop fasteners and are clearly shown being positioned substantially on the bottom side of the invention.

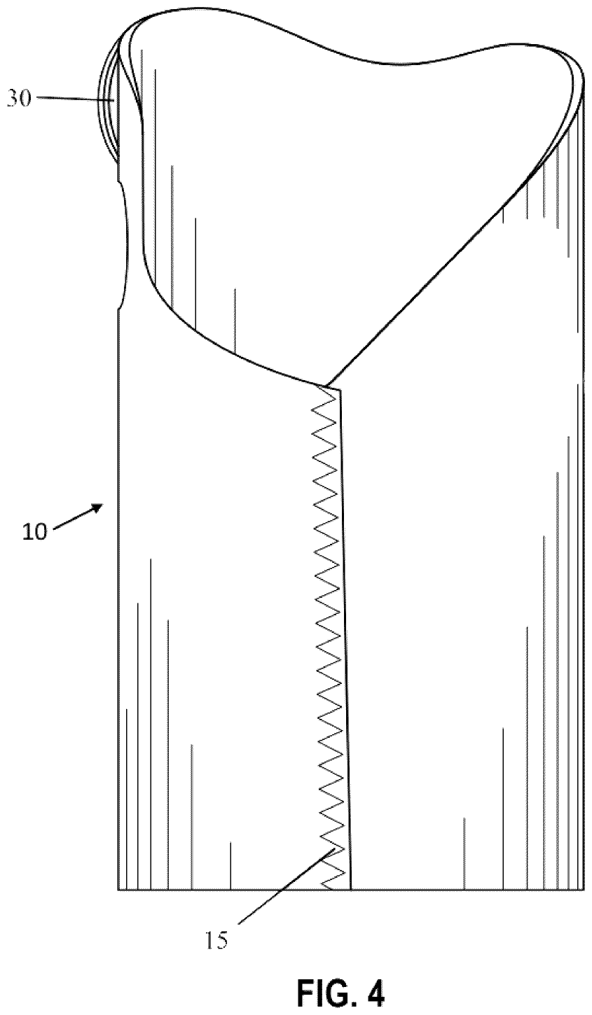

FIG. 5 is a left side view of an embodiment of the invention wherein the gauze material holder (40) is less visible than the gauze plug holder (45) and a cotton tip applicator holder (50) wherein the cylindrical holder (30) is holding a tin can embodied as a grease tin.

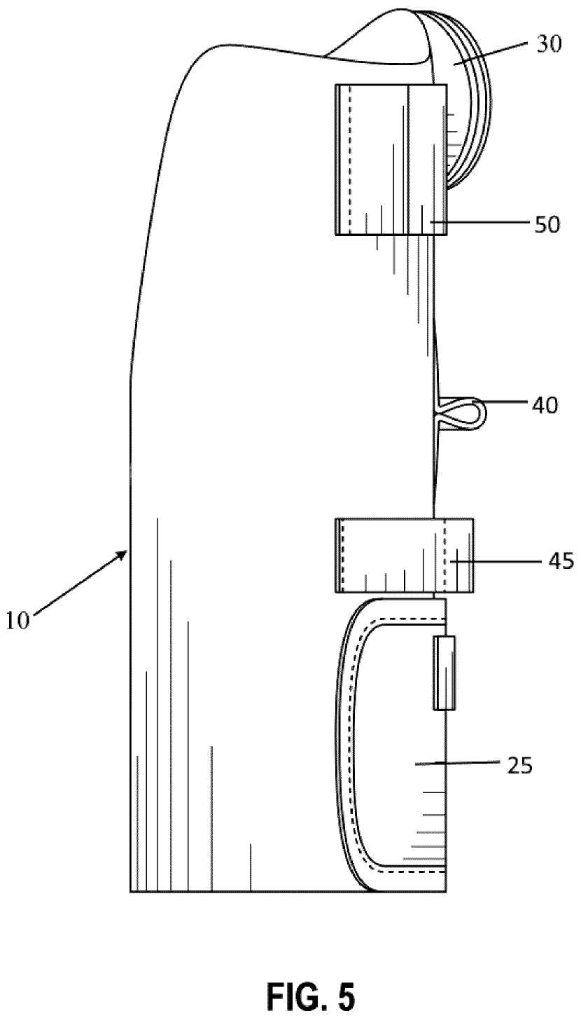

FIG. 6 is a right side view of an embodiment of the invention wherein the gauze material holder (40) is more visible than the gauze plug holder (45) and a cotton tip applicator holder (50) wherein the cylindrical holder (30) is holding a tin can embodied as a grease tin.

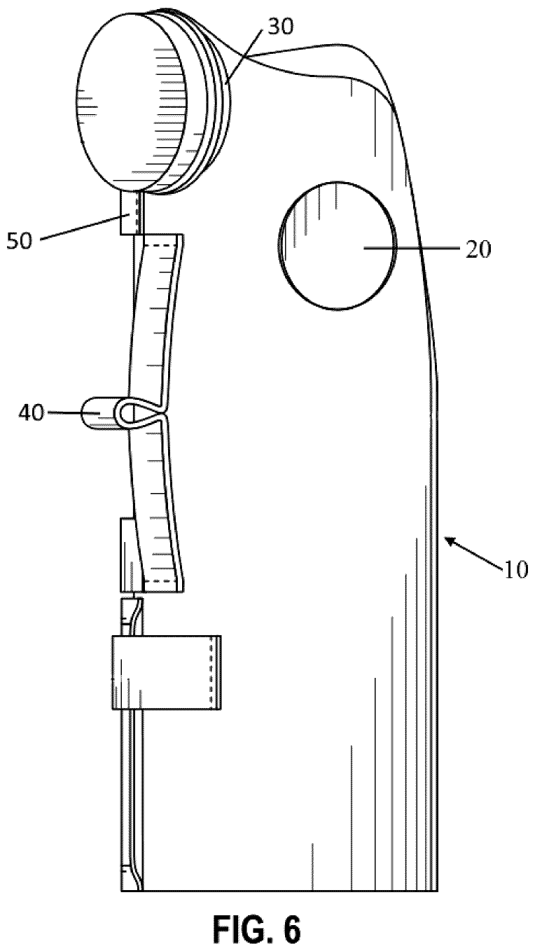

FIG. 7 is a front view of an embodiment of the present invention wherein the cylindrical holder (30) is holding a tin can embodied as a grease tin.

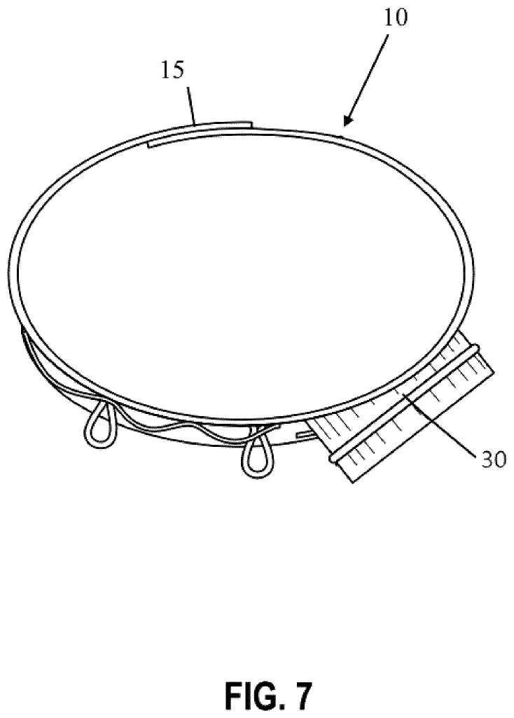

FIG. 8 is a back view of an embodiment of the present invention wherein the cylindrical holder (30) is holding a tin can embodied as a grease tin.

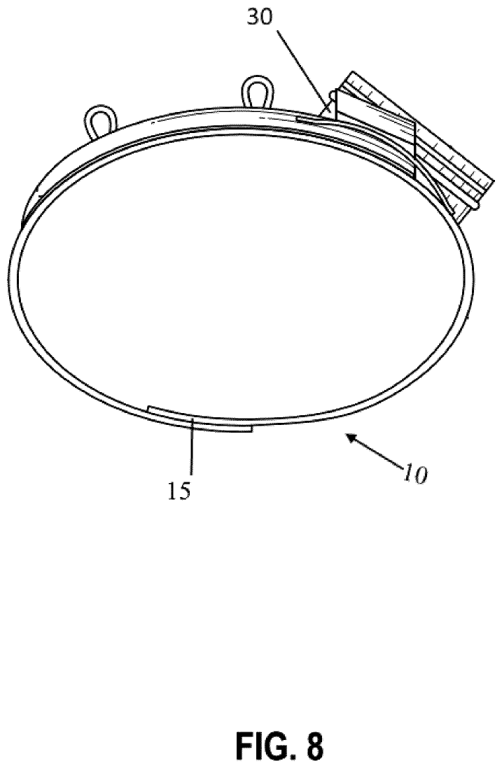

FIG. 9 is a top view of another embodiment of the invention wherein the fabric (10) is positioned with a user's hand to demonstrate positioning when wearing the fabric (10), wherein the fastening attachment mechanism (15) is in the form of hook-and-loop fasteners.

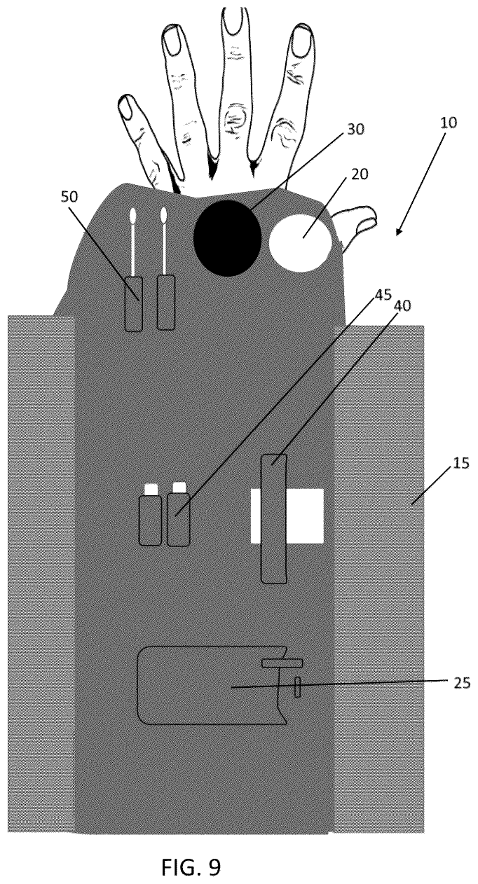

### DETAILED DESCRIPTION

Reference now will be made in detail to the embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

In accordance with the present invention, the present invention can be described as a fabric that assists coaches and teams in the field of athletic and competitive fighting, such as boxing, mixed martial arts, and other related sports. The fabric (10) will be of a generally elongated shape. The fabric (10) will have two sides. One side will be treated as an external side and the other side will be treated as an internal side. The fabric (10) is configured to wrap around the arm of a user so that the internal side is making contact with the user's arm directly and the external side is showing when worn by a user. In some embodiments, the fabric (10) will include at least one hole (20). This hole (20) will be positioned and configured so that when the user wears the fabric (10) around the user's arm, the thumb of the user's same arm that is wrapped in the fabric (10) fits through the hole (20).

The fabric (10) will include a fastening attachment mechanism (15). In some embodiments, the fastening attachment mechanism (15) will attach and hold the fabric (10) in place once the fabric (10) is positioned around the user's arm. In some embodiments, the fastening attachment mechanism (15) will attach and hold the fabric (10) in place once the fabric is so the thumb of the user is positioned in the hole. In some embodiments, the fastening attachment mechanism (15) will attach and hold the fabric (10) in place once the fabric (10) is positioned around the user's arm and the thumb of the user is positioned within the hole (20).

In some embodiments, the fastening attachment is positioned on the external side of the fabric (10). In some embodiments, the fastening attachment is positioned on the external side of the fabric (10) and at two different ends of the fabric (10). In some embodiments, the fastening attachment is a hook-and-loop fastener and positioned on the external side of the fabric (10). In some embodiments, the fastening attachment (15) is a hook-and-loop fastener and positioned on the external side of the fabric (10) wherein the hook section is at one end of the fabric (10) and the loop section is at the other end of the fabric (10). In some embodiments, the fastening attachment (15) is positioned on the bottom side of the fabric (10).

The fabric (10) will include a plurality of holders. In some embodiments these holders are positioned on the external side of the fabric (10). The plurality of holders are designed to hold specific instruments and tools that are relevant and helpful to an athletic fighting coach during intermission intervals or rest periods. The tools can include cotton swabs, enswells, grease tins, gauze, gauze plugs and more.

In some embodiments the fabric (10) will include a specific holder for only the enswell, which will be identified and referred to as an enswell holder(s) (25). In some embodiments the fabric (10) will include one or more enswell holders (25). The enswell is a metal device that is placed on ice and used as a cold compress and in some embodiments includes a handle. In some embodiments, one or more enswell holders (25) on the fabric (10) are configured to hold the enswell, wherein the handles of one or more enswells are exposed for easy access. In some embodiments the one or more enswell holders are thermally insulated keeping the enswell cold over time. In some embodiments, one or more enswell holders (25) are positioned on the top side of the fabric (10). In some embodiments the one or more enswell holders (25) are assisted with one or more elastic bands that help hold the one or more enswells In some embodiments there will be specifically no enswell holder (25) incorporated into the fabric (10).

The enswell holders (25) have several embodiments. The enswell holders (25) can be embodied as additional fabric sleeves. The enswell holders (25) can be embodied as a fabric sleeve and a protuberance. The protuberances can be part of the external surface of the fabric or a separate component integrated into the fabric. The enswell holders (25) can be embodied as plastic clips. The enswell holders (25) can be embodied as a plastic clip and a protuberance. The protuberance can be part of the external surface of the fabric (10) or a separate component integrated into the fabric (10). In some embodiments, the one or more enswell holders (25) are integrated with a cooling material.

The holders that hold grease tins and similar objects will be identified as cylindrical holders (30). In some embodiments, one or more cylindrical holders (30) are incorporated into the fabric (10). In some embodiments, one or more cylindrical holders (30) are incorporated into the top side of the fabric (10). In some embodiments, the cylindrical holder (30) is configured to only hold a certain size object such as, but not limited to a grease tin. In some embodiments, the cylindrical holder (30) is configured to hold a plurality of sizes. In some embodiments, the cylindrical holder (30) is configured into the fabric so upon use its position is between the thumb and the pointer finger of the user. In some embodiments, the tin cup is fixed to the sleeve by a velcro hook. In some embodiments, the tin cup is fixed to the sleeve by a velcro hook and can be placed anywhere on the sleeve.

The holders that hold cotton swabs in place will be identified as a cotton swab holder (35) and are fixed to the fabric (10). In some embodiments, one or more cotton swab holders (35) are incorporated into the fabric (10). In some embodiments, one or more cotton swab holders (35) are incorporated into the top side of the fabric (10). In some embodiments the cotton swab holder (35) will hold a single cotton swab. In some embodiments the cotton swab holder (35) will hold a plurality of cotton swabs. In some embodiments, the fabric (10) will include one or more gauze material holders (40). In some embodiments, the fabric (10) will include one or more gauze material holders (40) on the top side of the fabric (10).

The holders that hold gauze plugs in place will be identified as a gauze plug holder (45). In some embodiments, the fabric (10) will include one or more cause plug holders (45). In some embodiments the gauze plugs holder (45) will hold a single gauze plug. In some embodiments the gauze plugs holder (45) will hold a plurality of gauze plugs. In some embodiments the one or more gauze plug holders (45) are positioned on the top side of the fabric (10). In some embodiments the cotton swab holders (35) and/or the gauze plug holders (45) are made of elastic bands, holding the gauze and cotton plugs in place. In some embodiments, the elastic bands can be fixed onto the middle center of the sleeve. In some embodiments, the elastic bands can be fixed onto the middle center of the sleeve so upon use its position is below the user's thumb.

In some embodiments, one or more holders for a cotton tip applicator is incorporated into the fabric and are identified as cotton tip applicator holders (50). In some embodiments, the cotton tip applicator holder (50) is positioned on the fabric (10) so it is at the top side of the fabric (10). In some embodiments, the cotton tip applicator holder (50) is positioned on the fabric (10) so it is at the center of the fabric (10). In some embodiments, the cotton tip applicator holder (50) is positioned on the fabric (10) so it is at the center and top of the fabric (10). In some embodiments, the cotton tip applicator holder (50) consists of three small hole-like holders on the fabric (10). In some embodiments, the cotton tip applicator holder (50) consists of three small hole-like holders on the fabric (10) wherein the cotton tip applicator holder (50) is made of elastic stretch bands. The cotton tip applicator holder (50) is used to hold the cotton tip applicators by sliding the cotton tip applicators into the holes of the cotton tip applicator holders (50).

The fabric (10) is made of a variety of materials. In some embodiments, the fabric (10) is made completely of neoprene. In some embodiments, the fabric (10) is made partially of neoprene. In some embodiments, the fabric (10) is made completely of neoprene. The fabric (10) can be made of various types of material such as, but not limited to, gauze, cotton, polyester, cotton batting, leather, synthetic leather, wool, silk, rayon, nylon, taffeta, horse hair, adhesive, felt, othro gel, rubber, vinyl, acrylic and poly vinyl chloride polyurethane, polyethylene, polyborosiloxine, ethylvinylacetate, polyvinylchloride various types of foam including, but not limited to, open-cell foams, closed-cell foams, urethane foam, polyurethane foam, polyvinyl chloride (PVC) foam, memory foam, and other commonly known types of fabric padding, as well as various combinations of these materials.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device comprising of elongated fabric, wherein the fabric comprises of an external side, an internal side, a hole, with at least one fastening attachment mechanism, and a plurality of holders.

2. The device of claim 1, wherein the plurality of holders are disposed onto the external side of the fabric and the fastening mechanism.

3. The device of claim 1, wherein the plurality of holders comprises of at least one enswell holder.

4. The device of claim 1, wherein the plurality of holders comprises of an at least one cylindrical holder.

5. The device of claim 1, wherein the plurality of holders comprises of at least one enswell holder and an at least one cylindrical holder.

6. The device of claim 1, wherein the plurality of holders comprises of at least one enswell holder, at least one gauze holder, at least one cotton swab holder, at least one cylindrical holder, and at least one cotton tip applicator holder.

7. The device of claim 1, wherein the size and orientation of the fabric enables the internal side of the fabric to wrap around the arm of a user and wherein the size and orientation of the fabric enables the user to place their thumb.

8. The device of claim 1, wherein the fastening mechanism comprises of a hook-and-loop fastener.

9. The device of claim 8, wherein the hook-and-loop fastener is positioned on the external side of the fabric.

10. The device of claim 3, wherein enswell holder further comprises of a cooling material.

11. The device of claim 10, wherein the cooling material is lined within the external surface of the fabric.

12. The device of claim 1, wherein the fabric is made of neoprene.

13. The device of claim 1, wherein the fabric is partially made of neoprene.

14. The device of claim 3, wherein enswell holder further comprises a sleeve and a protuberance.

15. The device of claim 3, wherein enswell holder further comprises a plastic clip and a protuberance

16. The device of claim 4, wherein the at least one cylindrical holder (30) is configured to hold a tin.

17. The device of claim 4, wherein the at least one cylindrical holder (30) is configured to hold a tin of grease.

18. The device of claim 6, wherein the at least one cotton swab holder is positioned and oriented so that a cotton swab within the at least one cotton swab holder is held in a position parallel with the longest length of the fabric.

19. The device of claim 1, wherein the hole is positioned to fit around a user's thumb when the internal side of the fabric is wrapped around the arm of the user.
